(19) United States
(12) Patent Application Publication
(10) Pub. No.: US 2012/0123330 A1
(43) Pub. Date: May 17, 2012

(54) DEVICES AND METHODS FOR REMOVING OBSTRUCTIONS FROM A CEREBRAL VESSEL

(76) Inventors: John Miller, Redwood City, CA (US); Scott Wilson, Redwood City, CA (US)

(21) Appl. No.: 13/342,938

(22) Filed: Jan. 3, 2012

Related U.S. Application Data
(63) Continuation of application No. 11/490,843, filed on Jul. 21, 2006.

(51) Int. Cl. A61M 25/08; A61M 25/10

(52) U.S. Cl. 604/96.01; 604/528

(57) ABSTRACT

The present invention is directed to methods and devices for removing an obstruction. The system includes a support catheter which can be advanced into small and tortuous vessels. By advancing the support catheter nearer to the obstruction then can be achieved with conventional guide catheters, the support catheter reduces the likelihood vessel compression and collapse when pulling on the retrieval element.

### BACKGROUND OF THE INVENTION

The present invention is directed to devices and methods for removing obstructions from a cerebral vessel. In particular, the present invention may be useful in removing obstructions from the M1 or M2 Middle Cerebral Artery (MCA). Of course, the present invention may find uses in other parts of the cerebral vasculature and in other parts of the body.

Various problems can occur when attempting to remove hard, well organized and/or impacted clots or obstructions with a retrieval device in the Middle Cerebral Artery. Vessel compression and displacement can occur in the soft, flexible and relatively unsupported cerebral vasculature when force is applied to the clot and the retriever to dislodge the clot. For example, the proximal portion of the MCA and/or ICA can be particularly susceptible to vessel compression and collapse which may cause the vessel to fold up on itself making successful retraction difficult. Vessel compression and displacement can also produce excessive frictional forces between the vessel and the retriever.

Another problem which can occur is that the force exerted on an obstruction to dislodge and remove the obstruction may be exerted in a direction which is skewed relative to the longitudinal axis of the vessel. This problem may be particularly troublesome when the obstruction is in the MCA. The MCA arises from the top of the ICA and travels in a direction roughly 90 degrees from the ICA. The tortuous vasculature often results in the application of force to the obstruction which is not aligned with the axis of the vessel.

### SUMMARY OF THE INVENTION

In one aspect of the present invention, a support catheter is provided which can be advanced over an obstruction retrieval device and guided into the proximal portion of the MCA. Positioning the support catheter in the MCA at a position close to the retriever can provide a more effective transmission of force to the retriever. Positioning the support catheter in this manner may also provide a more axially oriented force than would be provided if the support catheter were not able to navigate to the MCA. Typical guide catheters, for example, are too stiff to navigate the tight bends and tortuosity of the distal ICA and the siphon. The support catheter of the present invention, on the other hand, is flexible enough to navigate the distal ICA and the siphon. The support catheter may also be large enough to allow for a microcatheter to be positioned between the support catheter and the retriever. The proximal portion of the support catheter may be somewhat stiff and rigid for optimized advancement and support.

The present invention is also directed to systems and methods for removing obstructions. The system may include a guide catheter having a lumen coupled to a suction source to aspirate the obstruction as is known. A microcatheter is positioned within the lumen of the guide catheter. The microcatheter is used to introduce the obstruction retriever into the obstruction. A support catheter is also provided which is advanceable over the retriever and optionally over the microcatheter as well. A distal end of the guide catheter is positioned proximal to the MCA. The support catheter may be advanced with the microcatheter and/or retriever as a system or may be introduced before introduction of the microcatheter and/or retriever.

The microcatheter is advanced through the guide catheter and into or distal to the obstruction. Withdrawal of the microcatheter exposes a portion of the retriever which is now in contact with the obstruction. If the retriever is deployed distal to the obstruction, the microcatheter and retriever are withdrawn together until the retriever engages the obstruction. The end of the support catheter is advanced to a position near the obstruction to provide the advantages described herein. The retriever may then be pulled proximally to dislodge and remove the obstruction. By advancing the support catheter nearer to the obstruction and retriever than would be possible with typical guide catheters, the problems concerning vessel compression and collapse discussed above may be reduced or avoided since the support catheter will provide a more axially directed force relative to the vessel. Another advantage of positioning the support catheter just proximal to the obstruction is that suction can be applied to the lumen of the support catheter to assist in removal of the obstruction.

These and other aspects of the present invention will become apparent from the following description. The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 shows a system for removing an obstruction from a vascular location.

FIG. 2 shows a retriever, microcatheter, support catheter and guide catheter in accordance with the present invention.

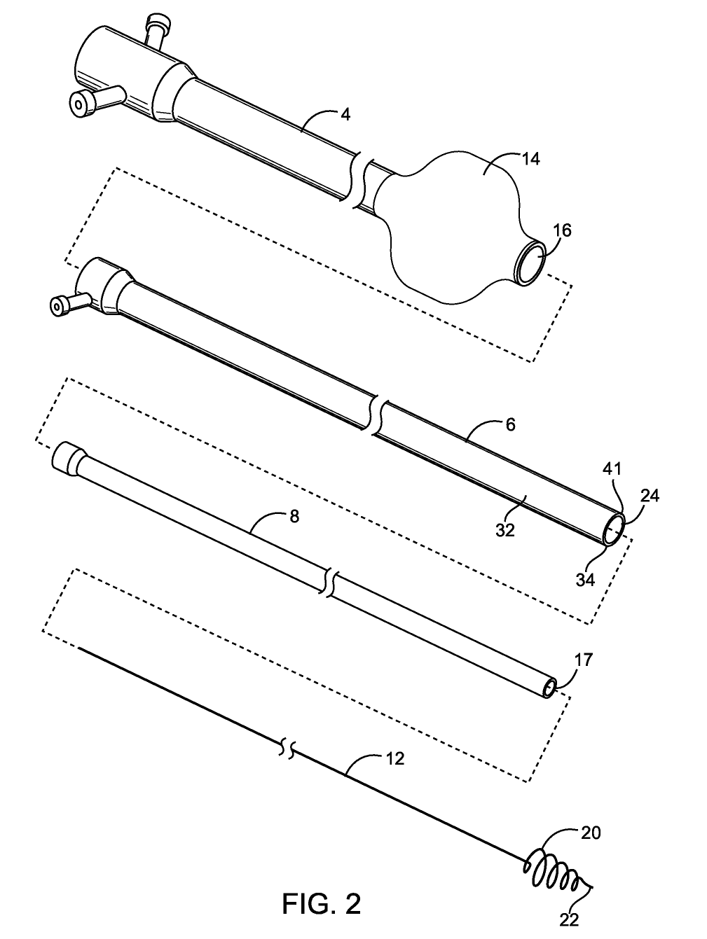

FIG. 3 shows a cross-sectional view of the construction of the support catheter

FIG. 4 shows removal of an obstruction in accordance with the present invention

### DESCRIPTION OF THE PREFERRED EMBODIMENTS

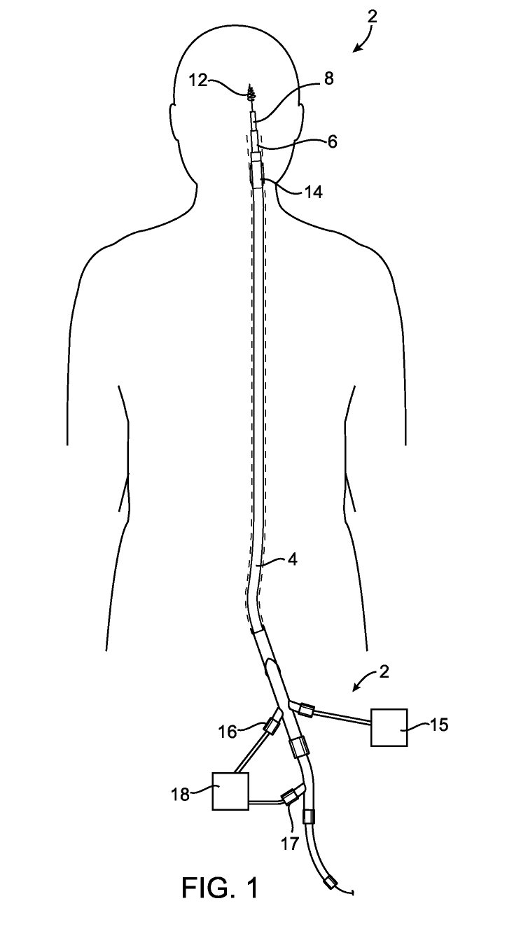

Referring to FIG. 1, a system 2 for removing an obstruction from a vascular location is shown. The system 2 is particularly useful for removing obstructions from the cerebral vasculature. The system 2 includes a guide catheter 4, a support catheter 6, a microcatheter 8, a guidewire and an obstruction retriever 12. The guide catheter 4 is advanced within the vasculature and guides the other catheters and devices through the larger vessels leading to the obstruction. The guide catheter 4 may have a balloon 14 which is coupled to a source of inflation fluid 15 and is inflated during the procedure to temporarily stop blood flow when removing the obstruction. The guide catheter 4 may have a lumen 16 and the support catheter 6 may have a lumen 17 which both may be coupled to a vacuum source 18 for aspirating the obstruction. Of course, the present invention may be practiced with one or more of the catheters without departing from various aspects of the invention. For example, the support catheter 6 could be used without the guide catheter 4 and/or microcatheter 8.

The guidewire is advanced into or distal to the obstruction and the microcatheter 8 is then advanced over the guidewire so that the microcatheter 8 is also positioned within or distal to the obstruction. The guidewire is then withdrawn and the retriever 12 is advanced through the microcatheter 8. The microcatheter 8 is then withdrawn to expose part of the retriever 12 within or distal to the obstruction and withdrawn, if necessary, to contact the obstruction. The retriever 12 may be any suitable device which engages and dislodges the obstruction. For example, the retriever 12 may have a self-expanding element 20 having a free end 22 so that the element 20 is essentially a single filament or wire. The system of the present invention is particularly useful when using small devices to engage and remove the obstruction. In particular, the system of the present invention is useful for retrievers 12 having an outer diameter of less than 0.021 inch and even less than 0.018 inch when collapsed and contained within the microcatheter 8. A suitable device which may be used for the retriever 12 is sold by Concentric Medical.

The support catheter 6 is sized to be advanced through the guide catheter 4 and over the retriever 12 and optionally over the microcatheter 8 as well. The support catheter 6 has a proximal portion which may be somewhat stiff and rigid for optimized advancement and support. A distal portion of the support catheter 6 is relatively flexible to navigate the tortuous vessels such as the distal ICA and the siphon. Although the support catheter 6 must be flexible enough to navigate these vessels, the support catheter 6 must also still be strong enough to prevent kinking and/or collapse during aspiration.

The support catheter 6 is sized to extend through the guide catheter 4 while optionally being large enough to accommodate the microcatheter 8. The support catheter 6 has a lumen 24 with a diameter of less than 0.065 inch along a distal portion 26 which extends at least 10 cm from a distal end 28. The microcatheter 8 may have an outer diameter of less than 0.060 inch, or even less than 0.050 inch, and may be within a range of about 0.025-0.060 inch along a distal portion 32 which extends for a length of at least 10 cm from a distal end 34. The lumen 17 of the microcatheter 8 may have a diameter of about 0.017 inch while the lumen of the support catheter 6 may have a diameter of about 0.040-0.065 inch.

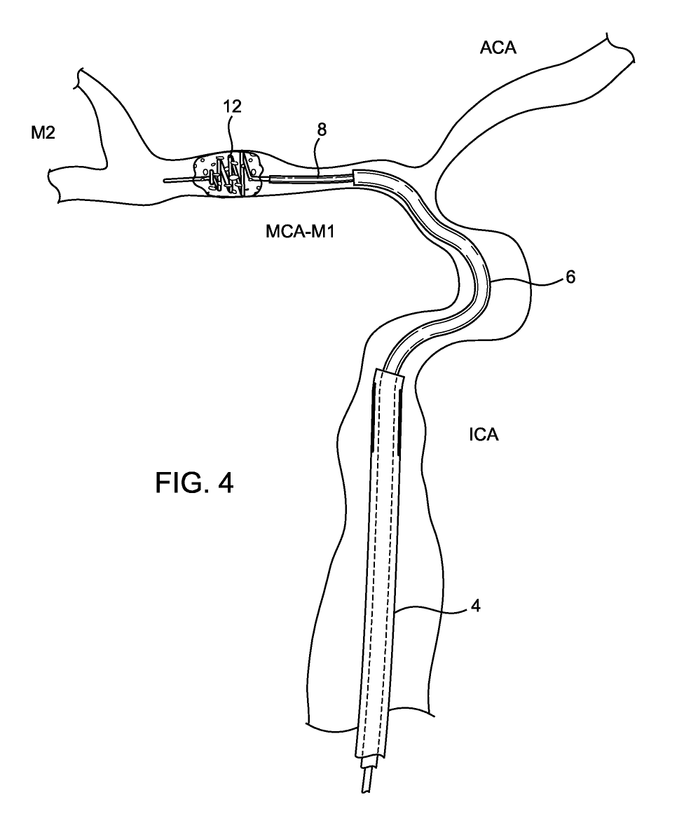

The support catheter 6 is advanced into small, torutuous vessels to provide support when dislodging and removing the obstruction with the retriever 12. The support catheter 6 is advanced further into the vasculature for obstructions in the MCA than typical guide catheters which are too stiff to navigate the distal ICA and siphon. By advancing the support catheter 6 nearer to the clot and retriever 12 than would be possible with typical guide catheters, the problems concerning vessel compression and collapse discussed above may be reduced or avoided. FIG. 4 shows the distal end 28 of the support catheter 6 advanced to a position near the retriever 12. Positioning the support catheter 6 near the retriever 12 may result in a more longitudinally directed force on the obstruction and may also reduce the problem of vessel compression and collapse compared to a typical guide catheter which cannot navigate the distal ICA and siphon.

The guide catheter 4 may be introduced into a femoral artery or other suitable access point. For example, the guide catheter 4 may be an 8Fr catheter with the balloon 14 being used to stop or reduce flow. The microcatheter 8 may be sized small enough to fit within the lumen 24 of the support catheter 6 or may be removed before introduction of the support catheter 6 if the microcatheter 8 is too large to be received by the support catheter 6. Of course, other guide catheters and microcatheters, including those without a balloon, may be used with the systems and methods of the present invention.

The design of the support catheter 6 is now described. The support catheter 6 may have a constant diameter or may have a tapered diameter along one or more sections. The following is a description of a support catheter 6 having a constant diameter. The support catheter 6 has an ID of about 0.060 inch and an OD of about 0.078 inch. The support catheter 6 has a body 40 having a distal tip formed of 25 D pebax and having a length of 1 mm to provide a flexible, atraumatic tip 41. A marker band is provided proximal to the tip 41 and is made of a material, such as platinum, which is readily visible to help identify the distal end of the catheter 6 when viewing the catheter 6 as is known in the art.

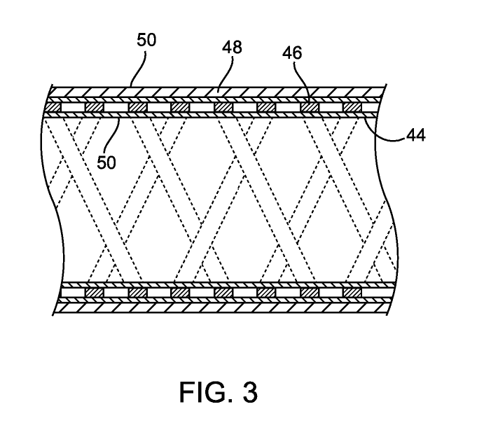

The catheter body 40 has increasing flexibility toward the distal end 28 of the catheter 6. Referring to FIG. 3, the catheter body 40 has a liner 44, a reinforcing layer 46 over the liner 44, an outer tube 48 and a coating 50 on the inner and/or outer surfaces. The liner 44, reinforcing layer 46 and outer tube 48 are fused together using a shrink tube (not shown) as is known in the art. The coating 50 may be a hydrophyllic coating used to reduce friction so that the various catheters and devices described herein may move smoothly against one another and within the vasculature.

The liner 44 may be an etched PTFE liner having a 0.060 inch ID and 0.0015 inch wall thickness. The reinforcing layer 46 may be a braided reinforcing layer made of stainless steel ribbon (0.001×0.005 inch) at 90 PIC. The outer tube 48 has a first section having a length of 15 cm from the marker and has a durometer of 25 D. The outer tube 48 also has a second section having a length of 5 cm and a durometer of 40 D, a third section having a length of 5 cm and a durometer of 55 D and a fourth section having a length of 35 cm and a durometer of 72 D with each section extending from the other toward the proximal end. The support catheter 6 may have additional increases in durometer toward the proximal end. Of course, the increases in stiffness may be accomplished in any other manner such as changing the reinforcing layer 46 to change the stiffness rather than changing the durometer of the outer tube 48.

The catheter 6 has a flexible distal portion 52 which is at least 10 cm long and even at least 15 cm long. The flexible distal portion 52 is designed to navigate the tight bends and tortuousity of the cerebral vessels and, in particular, to traverse the distal ICA and the siphon to reach the distal MCA while maintaining structural integrity throughout the procedure. By advancing the support catheter 6 nearer to the obstruction and the retriever 12 than would be possible with typical catheters, the problems concerning vessel compression and collapse discussed above may be reduced or avoided since the support catheter 6 will provide a more axially directed force relative to the vessel. Another advantage of positioning the support catheter 6 just proximal to the obstruction is that suction can be applied to the lumen of the support catheter 6 to assist in removal of the obstruction.

1-20. (canceled)

21. A method of positioning catheters in the cerebral vasculature, comprising the steps of:
providing a microcatheter and a support catheter, the microcatheter having a lumen and an outer diameter of 0.025-0.060 inch along a distal portion, the distal portion having a length of at least 10 cm from a distal end of the lumen, the support catheter being advanceable over the microcatheter, the support catheter having a distal portion having a lumen with a diameter of 0.040-0.065 inch and a length of at least 10 cm from a distal end;
positioning the microcatheter distal to the MCA; and
advancing the support catheter to at least a proximal portion of the middle cerebral artery.

22. The method of claim 21, wherein:
the advancing step is carried out after the positioning step, the support catheter being advanced over the microcatheter to at least the proximal portion of the middle cerebral artery.

23. The method of claim 21, wherein:
the providing step is carried out with the guide catheter having a balloon coupled to a source of inflation fluid.

24. The method of claim 21, wherein:
the providing step is carried out with an obstruction engaging element being provided, the obstruction engaging element being positioned within the lumen of the microcatheter.
